(19) United States
(12) Patent Application Publication
(10) Pub. No.: US 2017/0112395 A1
(43) Pub. Date: Apr. 27, 2017

(54) METHOD AND APPARATUS FOR ESTIMATING BLOOD PRESSURE

(71) Applicant: SAMSUNG ELECTRONICS CO., LTD., Suwon-si (KR)

(72) Inventors: Hyungjoo KIM, Seongnam-si (KR); Youngzoon YOON, Hwaseong-si (KR); Younggeun ROH, Seoul (KR)

(73) Assignee: SAMSUNG ELECTRONICS CO., LTD., Suwon-si (KR)

(21) Appl. No.: 15/096,415

(22) Filed: Apr. 12, 2016

(30) Foreign Application Priority Data
Oct. 27, 2015 (KR) 10-2015-0149731

(51) Int. Cl. A61B 5/021; A61B 5/024; A61B 5/0452; A61B 5/00

(52) U.S. Cl. CPC A61B 5/02125; A61B 5/7267; A61B 5/02416; A61B 5/0452

(57) ABSTRACT

A method of estimating a blood pressure is provided. The method of estimating blood pressure includes inputting physical characteristic information and blood pressure information of a subject, determining, among a plurality of groups classified according to hemodynamic characteristics, a group to which the subject belongs based on the physical characteristic information and the blood pressure information, detecting a bio-signal of the subject, extracting a plurality of features from the detected the bio-signal, and estimating a blood pressure corresponding to the extracted plurality of features and the determined group based on a learned blood pressure estimation algorithm.

### CROSS-REFERENCE TO RELATED APPLICATION

This application claims priority from Korean Patent Application No. 10-2015-0149731, filed on Oct. 27, 2015 in the Korean Intellectual Property Office, the disclosure of which is incorporated herein by reference in its entirety.

### BACKGROUND

1. Field

Apparatuses and methods consistent with exemplary embodiments relate to estimating blood pressure using an artificial neural network algorithm.

2. Description of the Related Art

A cuff-type blood pressure measuring device is bulky, inconvenient to carry, and inadequate for real-time sequential monitoring of blood pressure. Recently, a cuffless-type blood pressure measuring device has received significant attention.

The cuffless-type blood pressure measuring device indirectly measures blood pressure by using a source light. For example, by using photoplethysmography (PPG) a change in blood volume at a particular body area is measured, and a waveform of blood volume is analyzed, thus blood pressure is estimated. Another method is measuring a movement of skin on body epidermis due to a change in a blood vessel and estimating blood pressure.

A blood pressure estimation algorithm prepared by using a statistical method is applied to those methods of measuring blood pressure. A change in blood flow or a change in a movement of skin over the blood vessel is measured, and a correlation between features of these changes and an actually measured blood pressure is statistically determined to obtain the blood pressure estimation algorithm beforehand. In the method of estimating blood pressure, the features of a subject are matched to the blood pressure estimation algorithm.

However, a degree of accuracy of the estimation performed using the blood pressure estimation algorithm may be reduced depending on subjects.

### SUMMARY

Exemplary embodiments address at least the above problems and/or disadvantages and other disadvantages not described above. Also, the exemplary embodiments are not required to overcome the disadvantages described above, and may not overcome any of the problems described above.

One or more exemplary embodiments provide methods of estimating blood pressure by applying an artificial neural network algorithm for each of groups classified in accordance with a hemodynamic classification.

According to an aspect of an exemplary embodiment, there is provided a blood pressure estimation method including: inputting physical characteristic information and blood pressure information of a subject; determining, among a plurality of groups classified according to hemodynamic characteristics, a group to which the subject belongs based on the physical characteristic information and the blood pressure information; detecting a bio-signal of the subject; extracting a plurality of features from the detected bio-signal; and estimating a blood pressure corresponding to the extracted plurality of features and the determined group based on a learned blood pressure estimation algorithm.

The physical characteristic information may include sex, age, height and weight of the subject.

The determining comprises classifying the plurality of groups according to hemodynamic characteristics based on a heart rate, systolic blood pressure, diastolic blood pressure, cardiac output, total peripheral resistance change and pulse transit time.

The detecting of the bio-signal may be detecting a signal in accordance with a pulse wave speed change of light reflected off the subject.

The signal may be a photoplethysmography (PPG) signal or a pulse transit time signal.

The extracted plurality of features may include a systolic peak, a reflective peak, a systolic rising time, a reflective peak time and a period of the PPG signal.

The learned blood pressure estimation algorithm may correspond to a learned artificial neural network algorithm.

The estimating the blood pressure based on the neural network algorithm may include: learning an artificial neural network algorithm; and estimating the blood pressure by matching the extracted plurality of features to a hidden layer matrix of the learned artificial neural network algorithm.

The learning of the artificial neural network algorithm may include: inputting the plurality of features to an input layer of the artificial neural network algorithm; inputting a systolic blood pressure and a diastolic blood pressure of the blood pressure information to an output layer of the artificial neural network algorithm; and generating the hidden layer matrix having weights as well as thresholds of input values of the input layer on a hidden layer located between the input layer and the output layer.

According to another aspect of an exemplary embodiment, there is provided a method of estimating blood pressure including: inputting physical characteristic information and blood pressure information of a subject; detecting a bio-signal of the subject; extracting a plurality of features from the detected bio-signal; and estimating blood pressure by inputting the plurality of features, the physical characteristic information and the blood pressure information to a learned artificial neural network algorithm.

The estimating blood pressure may include: learning an artificial neural network algorithm; and estimating a blood pressure by matching the physical characteristic information and the plurality of features to a hidden layer matrix of the learned artificial neural network algorithm.

The inputting of the physical characteristic information and the blood pressure information may include determining, among a plurality of groups classified algorithmically according to hemodynamic characteristics, a group to which the subject belongs.

### BRIEF DESCRIPTION OF THE DRAWINGS

The above and/or other aspects will be more apparent by describing certain exemplary embodiments, with reference to the accompanying drawings, in which:

FIG. 1 is a schematic block diagram of an apparatus for estimating blood pressure, which is applied to a method of estimating blood pressure, according to an exemplary embodiment;

FIG. 2 is a flowchart of a method of estimating blood pressure, according to an exemplary embodiment;

FIG. 3 is a flowchart illustrating a classification of blood pressure with respect to hemodynamic characteristics, according to an exemplary embodiment;

FIG. 4 shows a graph of one cycle of a photoplethysmography (PPG) waveform according to an exemplary embodiment;

FIG. 5 is a diagram schematically illustrating an artificial neural network algorithm according to an exemplary embodiment;

FIG. 6 is a flowchart showing a method of learning an artificial neural network algorithm according to another exemplary embodiment; and

FIG. 7 is a flowchart schematically illustrating a method of estimating blood pressure, according to another exemplary embodiment.

### DETAILED DESCRIPTION

Exemplary embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the exemplary embodiments. However, it is apparent that the exemplary embodiments can be practiced without those specifically defined matters. Also, well-known functions or constructions are not described in detail since they would obscure the description with unnecessary detail.

As used herein, the term “and/or” includes any and all combinations of one or more of the associated listed items. Expressions such as “at least one of,” when preceding a list of elements, modify the entire list of elements and do not modify the individual elements of the list.

In the case where a position relationship between two items is described with the terms “on ˜” or “on the top of ˜,”, one item may be not only directly on the other item while being in contact with the other item but may also be on the other item without being in contact with the other item.

Throughout the specification, when a portion is connected to another portion, the case may include not only being directly connected but also being electrically connected with other elements therebetween. When a portion includes a composing element, the case may denote further including other composing elements without excluding other composing elements unless otherwise described. The terms “ . . . unit” or “module” may denote a unit performing one of specific function or movement and may be realized by hardware, software or a combination of hardware and software.

Throughout the specification, the term “consists of” or “includes” should not be interpreted as meaning that all of various elements or steps described in the specification are absolutely included, and should be interpreted as meaning that some of elements or steps may not be included or that additional elements or steps may be further included.

It will be understood that although the terms “first,” “second,” etc., may be used herein to describe various components, these components should not be limited by these terms. These terms are used only to distinguish one component from another.

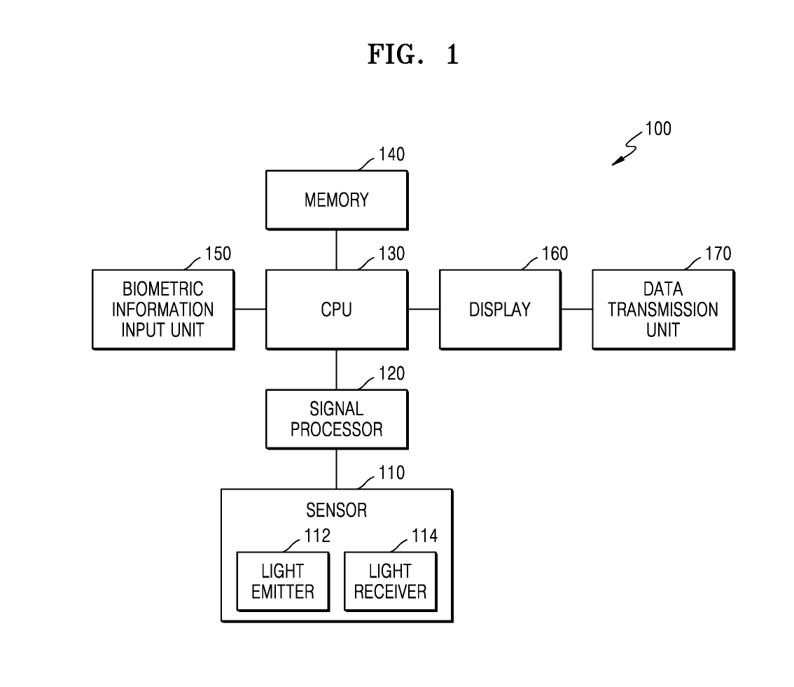

FIG. 1 is a schematic block diagram of an apparatus for estimating blood pressure 100, which is applied to a method of estimating blood pressure, according to an exemplary embodiment.

Referring to FIG. 1, the apparatus for estimating blood pressure 100 may include a sensor 110, a signal processor 120 for obtaining a bio-signal from signals detected by the sensor 110, a memory 140 storing a blood pressure estimation algorithm, and a central processing unit (CPU) 130 calculating blood pressure from the obtained bio-signal by using the blood pressure estimation algorithm. The apparatus for estimating blood pressure 100 may further include a display 160 displaying the calculated blood pressure, a biometric information input unit 150 inputting the biometric information to increase an accuracy of the blood pressure calculation and a data transmission unit 170 transmitting the calculated blood pressure to other devices. Blood pressure information of a subject may be input via the biometric information input unit 150.

The sensor 110 may include a light emitter 112 and a light receiver 114. The light emitter 112 may emit light to the subject. When the light reaches the subject, the light may be reflected from the subject and then travel back to the sensor 110. The light receive 114 may receive the reflected light and detect a difference between the emitted light and the received light. The light emitter 112 may include at least one of light emitting devices and the light receiver 114 may include at least one of light receiving devices.

The light emitting device may include a light emitting diode (LED) or a laser diode (LD). The light receiving device may include a photo diode or an image sensor such as a complementary metal-oxide semiconductor (CMOS) image sensor (CIS). The light receiving device may include a photo transistor (PTr). The light receiving device may be arranged to detect a signal change, in accordance with a change in blood flow, of light due to the light being scattered or reflected from a subject, that is, either from skin or a blood vessel of the subject.

However, the exemplary embodiment is not limited thereto. The sensor 110 may be a device for measuring a pulse transit time.

The signal processor 120 obtains a bio-signal from a signal detected by the sensor 110, and may remove a signal noise caused by outside lighting or ambient surroundings. The signal processor 120 may analyze an intensity change of a light signal detected by the sensor 110. The bio-signal may be obtained by analyzing a fluctuation of the light signal corresponding to a volume change of a blood vessel of the subject (for example, a radial artery in the wrist of the subject). In this case, the obtained bio-signal may be a transformed photoplethysmography (PPG) signal based on a correlation between a change in the analyzed light signal and a volume change of the blood vessel.

The signal processor 120 may include, for example, a waveform extractor extracting a waveform of one cycle from a signal input in real time and a data extractor sampling data from the waveform of one cycle either at regular time intervals or at irregular intervals set by a user. In addition, the signal processor 120 may further include a waveform selector.

A blood pressure estimation algorithm may be stored in the memory 140. In addition, a program for processing and controlling the signal processor 120 and the CPU 130 may be stored in the memory 140. Also, data to be input and output may be stored. In other words, measured results of the sensor 110 and the bio-signal obtained by the signal processor 120 may be stored. The memory 140 may store the bio-signal input in real time in a buffer memory. The CPU 130 may execute the blood pressure estimation algorithm that is stored in the memory 140 to estimate blood pressure.

The memory 140 may include at least one type of storage medium such as, for example, a flash memory type memory, a hard disk type memory, a multimedia card micro type memory, a card type memory (e.g., a secure digital (SD) memory and an extreme digital (XD) memory), a random access memory (RAM), a static random access memory (SRAM), a read-only memory (ROM), an electrically erasable programmable read-only memory (EEPROM), a programmable read-only memory (PROM), a magnetic memory, a magnetic disk, a photo disk, etc.

The CPU 130 may control an operation of the sensor 110 and measure blood pressure by using the blood pressure estimation algorithm. In other words, the CPU 130 may calculate blood pressure by using the blood pressure estimation algorithm from the bio-signal obtained through processing the signal measured by the sensor 110 in the signal processor 120. The CPU 130 may also control the memory 140, the display 160, the signal processor 120, and the biometric information input unit 150.

The CPU 130 may extract various features from the bio-signal, for example, a waveform of the PPG signal. In addition, the CPU 130 may estimate a blood pressure value by combining the extracted features and a matrix of the blood pressure estimation algorithm. In this case, the blood pressure value estimated by the CPU 130 may include a systolic blood pressure and a diastolic blood pressure.

The blood pressure value calculated in the CPU 130 may be displayed on the display 160. The display 160 may display the systolic blood pressure and the diastolic blood pressure.

The biometric information input unit 150 may receive physical characteristic information of the subject such as sex, age, height, and weight. The biometric information input unit 150 may also receive blood pressure information of the subject to increase an accuracy of blood pressure calculation. The blood pressure information may be already known information such as the systolic blood pressure and the diastolic blood pressure. The CPU 130 may determine the subject as belonging to one of predetermined groups in accordance with hemodynamic characteristics of the subject based on information input via the biometric information input unit 150, and may estimate blood pressure by applying a blood pressure estimation algorithm corresponding to the determined group. The groups in accordance with hemodynamic characteristics may be classified beforehand according to heartbeat, the systolic blood pressure, the diastolic blood pressure, cardiac output, a change in total peripheral resistance and pulse transit time.

The data transmission unit 170 may transmit a result analyzed in the CPU 130 to other external devices. The blood pressure value calculated and estimated in the CPU 130 may be output on the display 160, and the data transmission unit 170 may transmit the blood pressure value and the heart rate value to an external device such as a smart phone or a computer by using, for example, a communication device such as Bluetooth.

The external device may be the smart phone and the computer. Also, the external device may be medical equipment using analyzed blood pressure information, a printer to print out a result or a display device displaying an analyzed result. In addition, the external device may include various devices such as a tablet personal computer (PC), a personal digital assistant (PDA), a laptop, and other mobile or non-mobile computing devices.

The data transmission unit 170 may be connected to the external device via wire or wireless communication. For example, the data transmission unit 170 may communicate with the external device using various communication methods such as Bluetooth communication, Bluetooth low energy (BLE) communication, near field communication (NFC), wireless local area network (WLAN) communication, Zigbee communication, infrared data association (IrDA) communication, Wi-Fi direct (WFD) communication, ultra wideband (UWB) communication, Ant+communication and Wi-Fi communication.

The apparatus for estimating blood pressure 100 may further include a user interface (UI). The UI is an interface between a user and/or the external device, and may include an input unit and an output unit. Here, the user may be an object whose blood pressure is measured, that is, the subject; however, the user may be a person utilizing the apparatus for estimating blood pressure 100, such as a medical professional. Requisite information may be input to operate the apparatus for estimating blood pressure 100 through the UI and an analyzed result may be output. The UI may include, for example, a button, a connector, a keypad, a touch screen, etc., and may further include components such as a sound output unit and a vibration motor.

The apparatus for estimating blood pressure 100 may be a type of wearable device, a type of mobile phone such as a mobile smart phone type, or a type of tablet device. In other words, the apparatus for estimating blood pressure 100 may be mounted on the wearable device, the mobile phone such as the mobile smart phone, or the tablet device. In addition, the apparatus for estimating blood pressure 100 may be realized in such a form that it may be placed around a finger to measure blood pressure, that is, a finger tongs type apparatus.

For example, the apparatus for estimating blood pressure 100 may be realized as a device being worn by the subject, that is, in a shape of a wearable device. The wearable device may be realized as a wrist watch type, a bracelet type, or a wrist band type, and in addition, may be realized as various types such as a ring type, a glasses type, an earphone type, a headset type, or a hair-band type. In addition, only some components of the apparatus for estimating blood pressure 100, for example, the sensor 110 and the signal processor 120, may be worn by the subject.

The apparatus for estimating blood pressure 100 may be used as a device for estimating blood pressure and measuring the heart rate of the subject by being applied, for example, as a substitute for a sensor measuring a heart rate only, to a wrist watch-type wearable device which measures the heart rate by using a back side of a main body of the wrist watch. In addition, the apparatus for estimating blood pressure 100 may be applied to a smart phone, etc., utilizing the light emitting device and the CIS in order to estimate the blood pressure and to measure the heart rate of the subject.

Hereinafter, a method of estimating blood pressure according to an exemplary embodiment will be described with reference to FIG. 1.

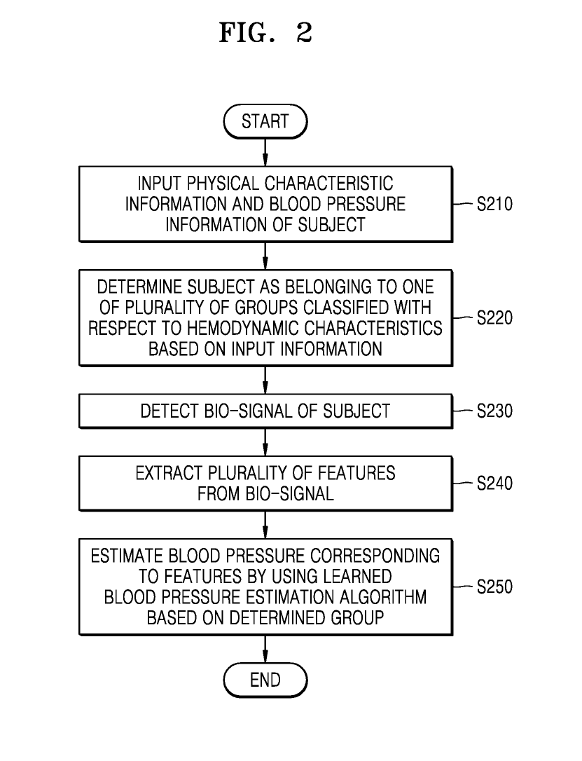

FIG. 2 is a flowchart of a method of estimating blood pressure according to an exemplary embodiment.

Referring to FIG. 2, physical characteristic information and blood pressure information of the subject may be input (operation S210). The physical characteristic information and the blood pressure information may be input via the biometric information input unit 150. The physical characteristic information of the subject may include at least sex, age, height and weight of the subject.

The blood pressure information may be the systolic blood pressure and the diastolic blood pressure.

When the physical characteristic information and the blood pressure information of the subject are already stored in the memory 140, the operation S210 may be omitted.

Then, the subject may be determined as belonging to one of a plurality of groups classified in accordance with hemodynamic characteristics based on the input information (operation S220). The plurality of groups may be predetermined based on the heartbeat, the systolic blood pressure, the diastolic blood pressure, the cardiac output, the change in total peripheral resistance, the pulse transit time, etc.

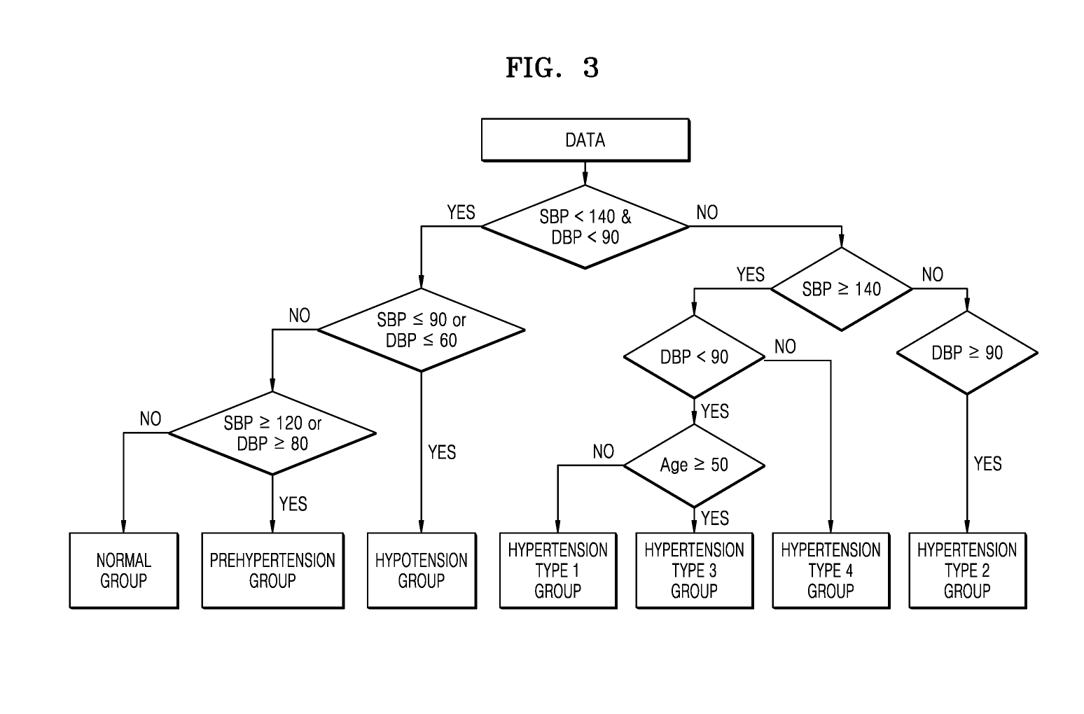

FIG. 3 is a flowchart of an example illustrating a classification of blood pressure with respect to hemodynamic characteristics.

In general, blood pressure may be classified into hypertension, normal blood pressure and hypotension. More errors may occur in estimating the hypertension than the normal blood pressure or hypotension, because there are many factors involved in indicating the hypertension.

A hypertension patient may have an individually different mechanism of blood pressure increase depending on age and causes of hypertension occurrence, and may be classified into three types in accordance with a hemodynamic mechanism as described below. A first type is an isolated systolic hypertension which occurs at a young age of about 17 to about 25. The systolic blood pressure increases more than the diastolic blood pressure, and the hypertension occurs due to a cardiac output and an aortic constriction, which are caused by excessive activity of a sympathetic nervous system.

A second type of hypertension has a normal systolic blood pressure and an increased diastolic blood pressure, and mainly occurs at an age of about 30 to about 50. The second type is related to obesity, and internal sodium and water content. The second type is an increase in the blood pressure due to hemodynamically improper cardiac output and elevated systemic vascular constriction.

A third type has an increase in the systolic blood pressure and normal or lower diastolic blood pressure, and mainly occurs at the age of about 55 and over. A pulse pressure increases due to an increase in the systolic blood pressure and a decrease in the diastolic blood pressure, which occur due to a decrease in an elasticity of blood vessel due to human aging. As such, hypertension patients may have blood vessels in different states in accordance with the hemodynamic mechanism. FIG. 3 diversely classifies hypertension patients sorted according to the described hemodynamic mechanism, but does not classify for subjects with the normal blood pressure and the hypotension blood pressure having little hemodynamic difference.

Referring to FIG. 3, the input blood pressure information may be classified, with respect to the systolic blood pressure (SBP) and the diastolic blood pressure (DBP), into a normal group, a prehypertension group, a hypotension group and type 1 to 4 hypertension groups.

Then, the bio-signal of the subject may be detected (operation S230). A laser beam may be emitted from the light emitter 112 to a radial artery. The light receiver 114 may receive light reflected from the radial artery. The bio-signal detected by the light receiver 114 may be a change in pulse wave speed. For example, the bio-signal may be an PPG signal.

Then, a plurality of features may be extracted from the detected bio-signal (operation S240).

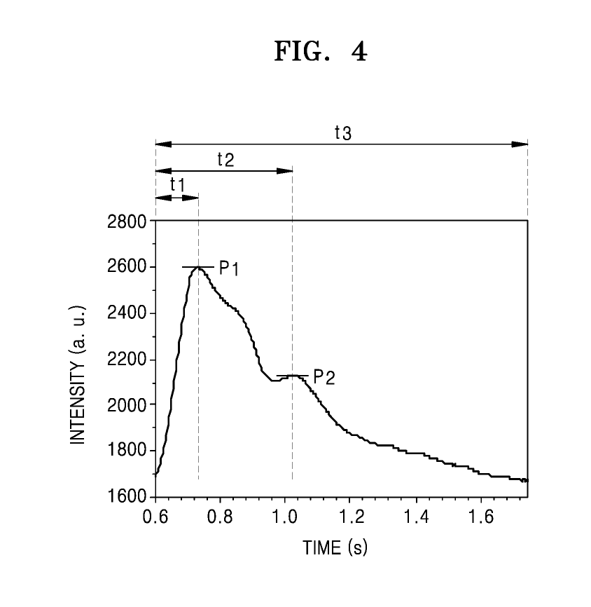

FIG. 4 shows a graph of one cycle of a waveform of the PPG signal.

Referring to FIG. 4, the plurality of features may exist in one cycle of the waveform. The plurality of features may include, for example, a systolic peak P1, a reflective peak P2, a systolic rising time t1, a reflective peak time t2 and a period t3.

Then, blood pressure corresponding to the features may be estimated by using a learned blood pressure estimation algorithm based on the determined group (operation S250).

The blood pressure estimation algorithm may be an artificial neural network (ANN) algorithm.

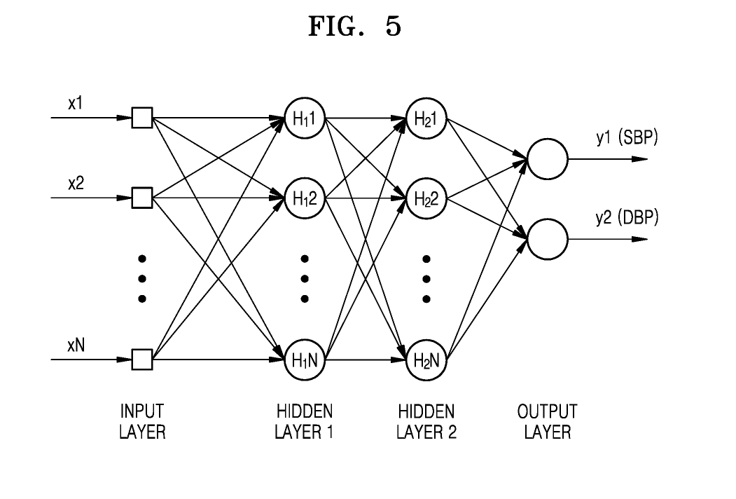

FIG. 5 is a diagram of an ANN algorithm.

Referring to FIG. 5, a hidden layer matrix structure may be prepared through machine learning such that the systolic blood pressure (SBP) y1 and the diastolic blood pressure (DBP) y2 may be output onto an output layer by corresponding features data x1, x2, . . . , xN input to an input layer to a plurality of hidden layers (e.g., hidden layer 1 and hidden layer 2). The hidden layer matrix may have weights and thresholds of input features, and output values, for example, the systolic blood pressure and the diastolic blood pressure may be estimated by matching input values to the hidden layer matrix.

When the ANN algorithm is constructed as in FIG. 5, the output data may be estimated by using the learned hidden layer matrix for an arbitrary input data.

Learned data may be stored in the form of the hidden layer matrix in the memory 140.

Physical characteristic information of the subject may be input along with the features to the input layer of the ANN algorithm. When the hemodynamic classification is performed with respect to the blood pressure of FIG. 3 and also with respect to the heartbeat, the cardiac output, the change in total peripheral resistance, and the pulse transit time of the subject, only the features may be input.

However, when the plurality of groups are hemodynamically classified in detail, the number of groups may increase and accordingly, a large amount of time and effort may be needed for an experiment (learning) to obtain the weights and the thresholds of the hidden layer of the ANN algorithm.

Thus, the number of groups may be simplified by classifying blood pressure in consideration of age basically as in FIG. 3 and inputting other biometric information along with features to the input layer. In other words, a classification with respect to general biometric information may be internally performed in the ANN algorithm.

On the other hand, a weight for a feature which is not input among features of the input layer becomes zero and may be excluded from the blood pressure estimation.

Hereinafter, a method of learning the ANN algorithm is described.

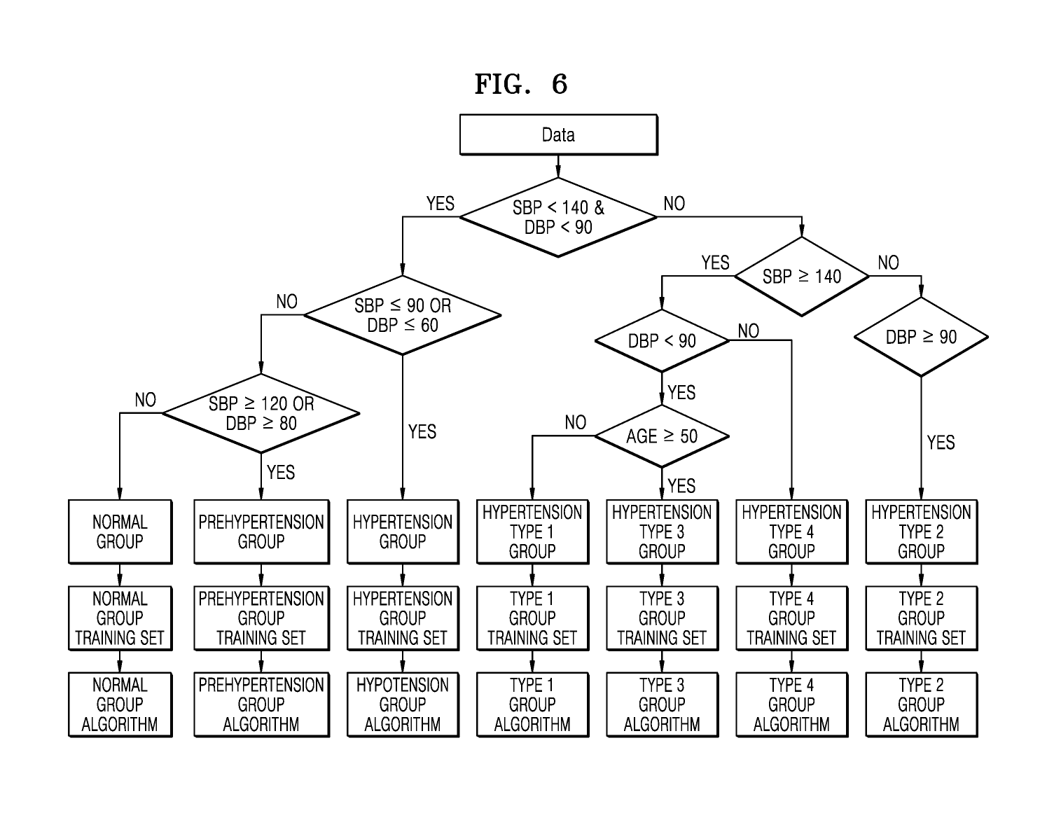

FIG. 6 is a flowchart showing a method of learning the ANN algorithm.

Referring to FIG. 6, a training set may be selected for each group classified in accordance with hemodynamic characteristics as described in relation to FIG. 3. The ANN algorithm for each group may be prepared through learning the ANN algorithm with respect to the selected training set.

The blood pressure estimation may be performed by using the learned ANN algorithm.

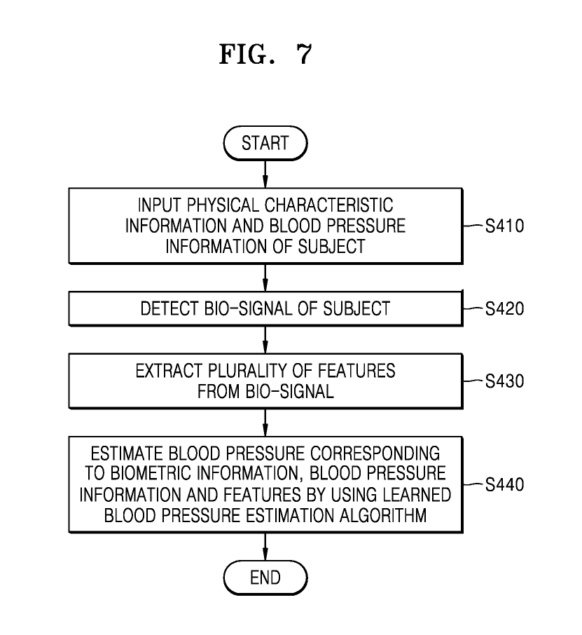

FIG. 7 is a flowchart schematically illustrating a method of estimating blood pressure according to another exemplary embodiment. The method is explained with respect to FIG. 1.

Referring to FIG. 7, biometric information of a subject may be input (operation S410). The biometric information may be input via the biometric information unit 150. The biometric information of the subject may include physical characteristic information such as sex, age, height and arm length of the subject, and may also include blood pressure information of the subject.

The blood pressure information may include the systolic blood pressure and the diastolic blood pressure.

When the biometric information of the subject are already stored in the memory 140, the operation S410 may be omitted.

Then, bio-signal of the subject may be detected (operation S420). A laser beam may be emitted from a light emitter 112 to a radial artery of the subject. A light receiver 114 may receive light reflected from the radial artery. The bio-signal detected by the light receiver 114 may be a PPG signal.

Then, a plurality of features may be extracted from the detected bio-signal (operation S430). The plurality of features may include, for example, a systolic peak P1, a reflective peak P2, a systolic rising time t1, a reflective peak time t2, and a period t3.

Then, the blood pressure corresponding to the physical characteristic information, the blood pressure information and the features may be estimated by using the learned blood pressure estimation algorithm (operation S440).

The blood pressure algorithm may be the ANN algorithm.

The physical characteristics information, the blood pressure information and the features may be input to an input layer (Input Layer of FIG. 5). The subject may be algorithmically classified as belonging to one of the plurality of groups, similar to the classification with respect to hemodynamic characteristics, according to the blood pressure information and age of the subject input to the input layer.

Output values, for example, the systolic blood pressure and the diastolic blood pressure may be estimated by matching the physical characteristic information, the blood pressure information and the features to the hidden layer matrix of the one of the plurality of groups.

Since blood pressure information, physical characteristic information and features of the subjects are input during the learning process of the ANN algorithm, a classification of input values such as the blood pressure information and the physical characteristic information of the subject may be internally performed, and blood pressure may be estimated based on the ANN algorithm which is internally classified in accordance with hemodynamic characteristics.

According to another exemplary embodiment, features, physical characteristic information, and blood pressure information of the subject may be input to the input layer, and the systolic blood pressure and the diastolic blood pressure of the blood pressure information may be input to the output layer for the learning of the ANN algorithm.

For the learning of the ANN algorithm, diversified data of subjects may be needed such that each of the feature, the physical characteristic information, and the blood pressure information may evenly contribute to the output information.

The learning of the ANN algorithm according to another exemplary embodiment may reduce a learning time compared with that of the learning of the ANN algorithm described in relation to FIG. 6.

The method of estimating blood pressure according to one or more exemplary embodiments may also be embodied as computer readable codes on a non-transitory computer readable recording medium. The non-transitory computer readable recording medium is any data storage device that can store data which can thereafter be read by a computer system. Examples of the non-transitory computer readable recording medium include read-only memory (ROM), random-access memory (RAM), CD-ROMs, magnetic tapes, floppy disks, optical data storage devices, etc. The non-transitory computer readable recording medium can also be distributed over network coupled computer systems so that the computer readable code is stored and executed in a distributive manner.

According to a method of estimating blood pressure, the ANN algorithm is performed with regard to persons belong to the same group divided by hemodynamic characteristics, and therefore an accuracy of the estimation of blood pressure is improved.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A method of estimating blood pressure, the method comprising:
inputting physical characteristic information and blood pressure information of a subject;
determining, among a plurality of groups classified according to hemodynamic characteristics, a group to which the subject belongs based on the physical characteristic information and the blood pressure information;
detecting a bio-signal of the subject;
extracting a plurality of features from the detected bio-signal; and
estimating a blood pressure corresponding to the extracted plurality of features and the determined group based on a learned blood pressure estimation algorithm.

2. The method of estimating blood pressure of claim 1, wherein the physical characteristic information comprises sex, age, height and weight of the subject.

3. The method of estimating blood pressure of claim 2, wherein the determining comprises classifying the plurality of groups according to the hemodynamic characteristics based on a heartbeat, a systolic blood pressure, a diastolic blood pressure, a cardiac output, a total peripheral resistance, and a pulse transit time.

4. The method of estimating blood pressure of claim 1, wherein the detecting the bio-signal comprises detecting a signal in accordance with a change in pulse wave speed of light reflected off the subject.

5. The method of estimating blood pressure of claim 4, wherein the signal is a photoplethysmography (PPG) signal or a pulse transit time signal.

6. The method of estimating blood pressure of claim 5, wherein the extracted plurality of features comprises a systolic peak, a reflective peak, a systolic rising time, a reflective peak time, and a period of the PPG signal.

7. The method of estimating blood pressure of claim 1, wherein the learned blood pressure estimation algorithm corresponds to a learned artificial neural network algorithm.

8. The method of estimating blood pressure of claim 7, wherein the estimating the blood pressure based on the learned neural network algorithm comprises:
learning an artificial neural network algorithm; and
estimating the blood pressure by matching the extracted plurality of features to a hidden layer matrix of the learned artificial neural network algorithm.

9. The method of estimating blood pressure of claim 8, wherein the learning the artificial neural network algorithm comprises:
inputting the extracted plurality of features to an input layer of the artificial neural network algorithm;
inputting a systolic blood pressure and a diastolic blood pressure of the blood pressure information to an output layer of the artificial neural network algorithm; and
generating the hidden layer matrix having weights and thresholds of input values of the input layer in a hidden layer located between the input layer and the output layer.

10. A method of estimating blood pressure, the method comprising:
inputting physical characteristic information and blood pressure information of a subject;
detecting a bio-signal of the subject;
extracting a plurality of features from the detected bio-signal; and
estimating a blood pressure by inputting the extracted plurality of features, the physical characteristic information, and the blood pressure information to a learned artificial neural network algorithm.

11. The method of estimating blood pressure of claim 10, wherein the inputting the physical characteristic information of the subject comprises inputting information including sex, age, height and weight of the subject.

12. The method of estimating blood pressure of claim 10, wherein the detecting the bio-signal comprises detecting a signal in accordance with a change in a pulse wave speed of light reflected off the subject.

13. The method of estimating blood pressure of claim 12, wherein the signal is a photoplethysmography (PPG) signal or a pulse transit time signal.

14. The method of estimating blood pressure of claim 13, wherein the extracted plurality of features comprises a systolic peak, a reflective peak, a systolic rising time, a reflective peak time and a period of the PPG signal.

15. The method of estimating blood pressure of claim 10, wherein the estimating the blood pressure comprises:
learning an artificial neural network algorithm; and
estimating a blood pressure by matching the physical characteristic information, the blood pressure information, and the extracted plurality of features to a hidden layer matrix of the learned artificial neural network algorithm.

16. The method of estimating blood pressure of claim 15, wherein the inputting the physical characteristic information and the blood pressure information comprises determining, among a plurality of groups classified algorithmically according to hemodynamic characteristics, a group to which the subject belongs.

17. The method of estimating blood pressure of claim 16, wherein the learning the artificial neural network algorithm comprises:
inputting the physical characteristic information, the blood pressure information, and the extracted plurality of features to an input layer of the neural network algorithm;
inputting a systolic blood pressure and a diastolic blood pressure of the blood pressure information to an output layer of the neural network algorithm; and
generating the hidden layer matrix having weights and thresholds of input values of the input layer in a hidden layer between the input layer and the output layer.

18. An apparatus for estimating blood pressure comprising:
a biometric information input unit configured to input physical characteristic information and blood pressure information of a subject;
a sensor configured to emit light to the subject to be reflected from the subject and detect a signal from the reflected light;
a signal processor configured to obtain a bio-signal from the detected signal;
a memory configured to store a blood pressure estimation algorithm; and
a central processing unit (CPU) configured to determine, among a plurality of groups classified according to hemodynamic characteristics, a group to which the subject belongs based on the physical characteristic information and the blood pressure information, extract a plurality of features from the bio-signal, and execute the blood pressure estimation algorithm to estimate a blood pressure corresponding to the extracted plurality of features and the determined group.
